(19) United States
(12) Patent Application Publication
(10) Pub. No.: US 2025/0006327 A1
(43) Pub. Date: Jan. 2, 2025

(54) AUTONOMOUS GENERATION OF ACCURATE HEALTHCARE SUMMARIES

(71) Applicant: NEC Laboratories America, Inc., Princeton, NJ (US)

(72) Inventor: Christopher Malon, Fort Lee, NJ (US)

(21) Appl. No.: 18/746,884

(22) Filed: Jun. 18, 2024

Related U.S. Application Data
(60) Provisional application No. 63/524,276, filed on Jun. 30, 2023.

(51) Int. Cl. G16H 15/00; G16H 50/20

(52) U.S. Cl. CPC G16H 15/00; G16H 50/20

(57) ABSTRACT

Systems and methods for autonomous generation of accurate healthcare summaries. Relevant healthcare questions can be predicted based on a preceding context by employing a fine-tuned transformer model. Answers to the relevant healthcare questions can be predicted by employing an extractive question answering model and utilizing extracted healthcare data from a healthcare data record to obtain predicted healthcare answers. Complete sentences can be synthesized, with artificial intelligence (AI), from the predicted healthcare answers and the relevant healthcare questions to obtain healthcare summary sentences. A healthcare technical report can be generated autonomously with AI from the healthcare summary sentences to assist with a decision making of a healthcare professional.

### RELATED APPLICATION INFORMATION

This application claims priority to U.S. Provisional App. No. 63/524,276, filed on Jun. 30, 2023, incorporated herein by reference in its entirety.

### BACKGROUND

### Technical Field

The present invention relates to healthcare data analysis and more particularly to autonomous generation of accurate healthcare summaries.

### Description of the Related Art

Natural language processing (NLP) has been developing at a rapid pace allowing artificial intelligence (AI) models to generate human-like text through text generative models. However, there has been some difficulty in ensuring that the generated text is factually correct especially for models that are trained on a generalized dataset. These models can struggle to generate factually relevant text based on a specific input containing distinct data.

### SUMMARY

According to an aspect of the present invention, a computer-implemented method for autonomous generation of accurate healthcare summaries is provided, including, predicting relevant healthcare questions based on a preceding context by employing a fine-tuned transformer model, predicting answers to the relevant healthcare questions by employing an extractive question answering model and utilizing extracted heathcare data from a healthcare data record to obtain predicted healthcare answers, synthesizing, with artificial intelligence (AI), complete sentences from the predicted healthcare answers and the relevant healthcare questions to obtain healthcare summary sentences, and generating a healthcare technical report autonomously from the healthcare summary sentences to assist with a decision making of a healthcare professional.

According to another aspect of the present invention, a system to autonomously generate accurate healthcare summaries is provided, including, a memory, one or more processor devices in communication with the memory to predict relevant healthcare questions based on a preceding context by employing a fine-tuned transformer model, predict answers to the relevant healthcare questions by employing an extractive question answering model and utilizing extracted healthcare data from a healthcare data record to obtain predicted healthcare answers, synthesize, with artificial intelligence (AI), complete sentences from the predicted healthcare answers and the relevant healthcare questions to obtain healthcare summary sentences, and generate a healthcare technical report autonomously from the healthcare summary sentences to assist with a decision making of a healthcare professional.

According to yet another aspect of the present invention, a non-transitory computer program product comprising a computer-readable storage medium including program code to autonomously generate accurate healthcare summaries is provided, wherein the program code when executed on a computer causes the computer to perform predicting relevant healthcare questions based on a preceding context by employing a fine-tuned transformer model, predicting answers to the relevant healthcare questions by employing an extractive question answering model and utilizing extracted healthcare data from a healthcare data record to obtain predicted healthcare answers, and synthesizing, with artificial intelligence (AI), complete sentences from the predicted healthcare answers and the relevant healthcare questions to obtain healthcare summary sentences, and generating a healthcare technical report autonomously from the healthcare summary sentences to assist with a decision making of a healthcare professional.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a flow diagram illustrating a high-level overview of a computer-implemented method for autonomous generation of accurate healthcare summaries, in accordance with an embodiment of the present invention;

FIG. 2 is a block diagram illustrating an overview of a computing device that may be configured to autonomously generate accurate healthcare summaries, in accordance with an embodiment of the present invention;

FIG. 3 is a block diagram illustrating a software program for autonomous generation of accurate healthcare summaries, in accordance with an embodiment of the present invention;

FIG. 4 is a block diagram illustrating a practical application of the computer-implemented method for autonomous generation of accurate healthcare summaries, in accordance with an embodiment of the present invention; and

FIG. 5 is a block diagram illustrating an overview of deep learning neural networks, in accordance with an embodiment of the present invention.

### DETAILED DESCRIPTION OF PREFERRED EMBODIMENTS

In accordance with embodiments of the present invention, systems and methods are provided for autonomous generation of accurate healthcare summaries.

In an embodiment, a healthcare technical report can be generated autonomously from healthcare summary sentences to assist with a decision making of a healthcare professional. Complete sentences can be synthesized with artificial intelligence (AI) from predicted healthcare answers and relevant healthcare questions to obtain healthcare summary sentences. Answers to the relevant healthcare questions can be predicted by employing an extractive question answering model that utilizes extracted healthcare data from a healthcare data record to obtain predicted healthcare answers. The relevant healthcare questions can be predicted based on a preceding context by employing a fine-tuned transformer model.

In another embodiment, an artificial intelligence (AI) assistant can be trained with the extracted healthcare data and corresponding textual prompts for a patient to assist a doctor in generating a medical summary for a patient.

Prior art solutions impute the most likely description of a particular condition by generating summaries directly without first generating relevant healthcare questions which may not be grounded in information pertaining to a given task. For example, a doctor can be presented with a patient exhibiting symptoms of tuberculosis that is also relevant to pneumonia. Generating summaries for both illnesses without asking relevant healthcare questions can lead to confusion and would be detrimental to the patient's health. Additionally, without asking relevant healthcare questions, the generated summary can overlook a patient's predispositions (e.g., hypertension predisposition, diabetes predisposition, etc.) and other inherited conditions that can be included in the patient's healthcare record.

The present embodiments can improve accuracy of predicted healthcare reports and summaries by first generating relevant healthcare questions and predicting answers to the relevant healthcare questions by employing an extractive question answering model. Additionally, the present embodiments can also interact with a decision-making entity (e.g., doctor) to autonomously generate summaries that can be inferred from the data which can be validated by the doctor.

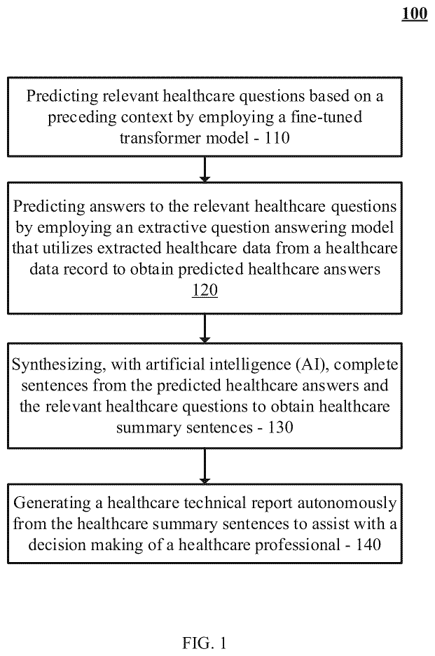

Referring now in detail to the figures in which like numerals represent the same or similar elements and initially to FIG. 1, a high-level overview of a computer-implemented method for autonomous generation of accurate healthcare summaries is illustratively depicted in accordance with one embodiment of the present invention.

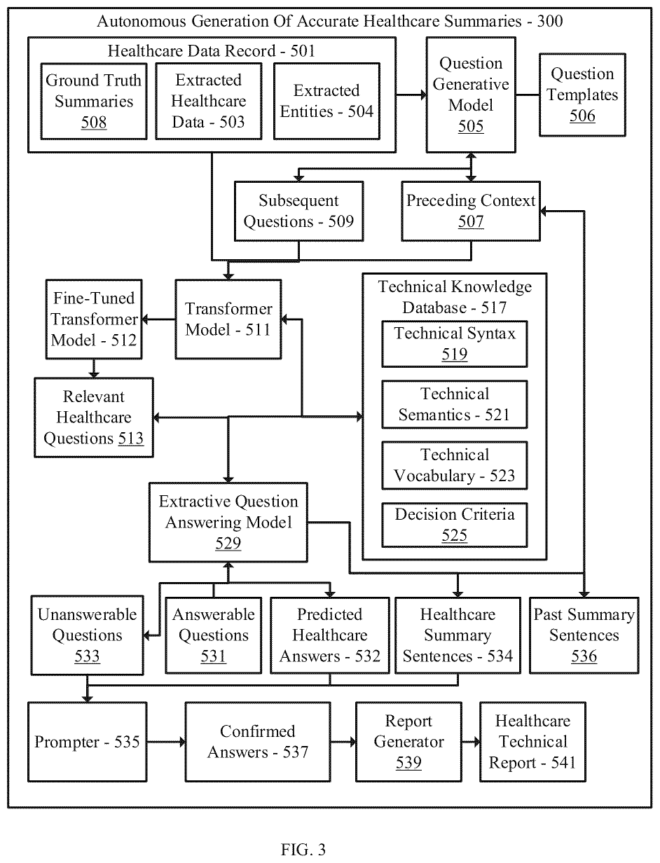

In an embodiment, relevant healthcare questions 513 (shown in FIG. 3) can be predicted based on a preceding context 507 (shown in FIG. 3) by employing the fine-tuned transformer model 512 (shown in FIG. 3). Predicted healthcare answers 532 (shown in FIG. 3) to the relevant healthcare questions 513 (shown in FIG. 3) can be predicted by employing an extractive question answering model 529 (shown in FIG. 3). Complete sentences can be synthesized, with artificial intelligence (AI), from the predicted healthcare answers 532 and the relevant healthcare questions 513 to obtain healthcare summary sentences 534 (shown in FIG. 3). A healthcare technical report 541 (shown in FIG. 3) can be generated autonomously with AI from the healthcare summary sentences 534 to assist with a decision making of a healthcare professional 601.

In block 110, relevant healthcare questions 513 can be predicted based on a preceding context by employing the fine-tuned transformer model 512.

In an embodiment, relevant healthcare questions 513 can be predicted based on a preceding context 507 by employing the fine-tuned transformer model 512. The preceding context 507 can be generated from ground truth summaries 508 (shown in FIG. 3) that can be obtained from a healthcare data record 501 by employing a question generative model 505.

A description of the question generative model 505 and how it can utilize preceding context 507 to generate subsequent questions 509 (shown in FIG. 3) will now be shown in accordance with an embodiment of the present invention.

The question generative model 505 can generate subsequent questions 509 by utilizing ground truth summaries 508. In an embodiment, the question generative model 505 can be an AI text generation model that can be trained to generate subsequent questions 509 that can be answered with given sentences from the healthcare data record 501.

In another embodiment, the question generative model 505 can convert subsequent statements in ground truth summaries 508 to subsequent questions 509 by filling in a question template 506 (shown in FIG. 3) with extracted entities 504 (e.g., diagnoses or treatment names found in the ground truth summaries 508). The question template 506 can be created digitally and can be edited through a computer by the decision-making entity 628. For example, the question template 506 can be “Does the patient have <blank>?” In another embodiment, the question template 506 can be sent through a network. In another embodiment, the question template 506 can be obtained through computer memory. The question generative model 505 can judge the relevancy of the extracted healthcare data 503 to the question templates 506 based on rules created by the decision-making entity 628. For example, the rule can be that an extracted entity 504 can be relevant to the question template 506 “Does the patient have <blank>?”

The question generative model 505 can be a text-to-text transfer transformer (T5) model, generative pre-trained transformer (GPT™), pointer-generator networks or bidirectional encoder representations from transformers (BERT) models. Other AI text generation model structures are contemplated. The question generative model 505 can be trained in reverse with an extractive question answering dataset such as the Stanford® Question Answering Dataset (SQuAD) to predict questions from sentences that can answer them. Other extractive question answering datasets are contemplated.

A description of the healthcare data record 501 will now be shown in accordance with an embodiment of the present invention.

The healthcare data record 501 can include extracted healthcare data 503 such as current data and historical data. The current data can be information related to a particular task. For example, in the healthcare setting, the current data can be the patient's vital data obtained during the clinical visit (e.g., current medical history) such as blood pressure, temperature, blood oxygen saturation levels, body scans, etc. The historical data can be past recorded information related to the particular task. For example, the historical data can be the patient's past vital data (e.g., past medical history) obtained from a past clinic visit which can include family healthcare history such as health predispositions and other known illnesses of family members. The healthcare data record can include a textual prompt. The textual prompt can be information that pertains to a specific aspect of the particular task. For example, the textual prompt can be related to current symptoms (e.g., chief complaint) that are being experienced by the patient. The healthcare data record can also include ground truth summaries 508. Ground truth summaries 508 can be texts that describe facts. For example, a ground truth summary can be “A 51-year-old white male diagnosed with PTLD in latter half of 2007.”

In an embodiment, the healthcare data record 501 can include structured data and unstructured data. Structured data can include information that is organized in a particular way such as tables, graphs, charts, etc. Unstructured data can include information that is not organized in a particular way such as prose. In another embodiment, structured and unstructured data can also include information that is presented according to technical conventions in a particular field. For example, abbreviations and technical terminologies in “No cp/sob/n/v/diarrhea” in the medical field can mean “cp” as cerebral palsy, “sob” as shortness of breath, “n” as nausea, “v” as vomiting.

In another embodiment, a healthcare data record 501 can include clinical images and test results which can correspond with technical vocabulary 523. For example, an X-ray of a person can show symptoms of tuberculosis.

In another embodiment, the technical conventions can include technical syntax 519, technical semantics 521, and technical vocabulary 523 that can be adopted by a particular entity such as hospitals, universities, companies, etc. For example, one hospital might have an abbreviation for triaging their patients as low priority as “lp”, medium priority as “mp,” high priority as “hp,” clinically dead as “cd;” while another hospital might have different abbreviations or classification for the same concepts: low priority as green, medium priority as yellow, high priority as red, and clinically dead as black. The technical syntax 519 can be the way information is presented (e.g., “lp”). The technical semantics 521 can be the context of the information presented (e.g., “lp”=low priority). The technical vocabulary 523 can store the technical syntax 519, technical semantics 521 and other specialized words in a particular field (e.g., tuberculosis).

A description of the fine-tuned transformer model 512 and how the fine-tuned transformer model 512 can predict relevant healthcare questions 513 will now be shown in accordance with an embodiment of the present invention.

A transformer model 511 can be fine-tuned with the extracted healthcare data 503, the preceding context 507, and the subsequent questions 509. The transformer model 511 can be a large language model (LLM) such as Stanford® Alpaca, GPT™, T5 or BERT. Other LLMs are contemplated. The transformer model 511 can be fine-tuned by minimizing a loss function to obtain a fine-tuned transformer model 512. In another embodiment, the transformer model 511 can be fine-tuned with past medical history, the chief complaint and current healthcare data.

In an embodiment, the fine-tuned transformer model 512 can be configured to predict relevant healthcare questions 513 based on preceding context 507. The fine-tuned transformer model 512 can predict relevant healthcare questions 513 by taking preceding context 507, that can include past summary sentences 536 (shown in FIG. 3), as input to decide what question to ask next.

In another embodiment, fine-tuned transformer model 512 can predict questions based on the chief complaint and past medical history from the extracted healthcare data 503 as the input textual prompts.

In block 120, answers 532 can be predicted from the relevant healthcare questions 513 by employing an extractive question answering model 529.

In an embodiment, the extractive question answering model 529 can be a large language model (LLM) such as Stanford® Alpaca, GPT™, T5 or BERT. Other LLMs are contemplated. In another embodiment, the extractive question answering model 529 can be trained with the technical knowledge database 517. By training with the technical knowledge database 517, the extractive question answering model 529 can identify extracted healthcare data 503 based on a learned technical syntax 519 relevant to a textual prompt. Additionally, by training with the technical knowledge database 517, the extractive question answering model 529 can determine the context of the extracted healthcare data 503 based on the learned technical semantics 521 relevant to the textual prompt. Further, by training with the technical knowledge database 517, the extractive question answering model 529 can determine the learned technical meaning of the extracted healthcare data 503 based on learned technical vocabulary 523. The technical vocabulary 523 can include answer contexts that involve abbreviations. In another embodiment, the extractive question answer model 529 can be trained to pair answer contexts that involve abbreviations with questions that do not involve abbreviations (e.g., spelled out words).

If the extractive question answering model 529 cannot answer the relevant question 513, then the relevant question can be an unanswerable question 533. In another embodiment, to determine whether a relevant question 513 is answerable, the technical knowledge database 517 can be queried to determine whether the fine-tuned transformer model 512 already learned the text included in the extracted healthcare data 503 and the textual prompt which can be stored as a decision criterion 525. If there is no corresponding decision criterion 525, then the relevant question 513 can be an unanswerable question 533. Otherwise, the relevant question 513 can be an answerable question 531.

In an embodiment, the extractive question answering model 529 can predict an answer 532 by using the relevant healthcare questions 513 and extracted healthcare data 503 as inputs to convert the relevant question 513 into a complete sentence. The extractive question answering model 529 can be trained on an extractive question answering dataset such as the Stanford® Question Answering Dataset (SQuAD). A complete sentence can be a statement that contains a subject and a verb.

In another embodiment, answers 532 to relevant healthcare questions 513 can be predicted from a healthcare data record 501 that can include clinical images and test results which can correspond with technical vocabulary by employing a visual question answer generative model. A visual question answer generative model can predict answers 532 based on the healthcare data record 501. The visual question answer generative model can be a visual-text generative model such as a large language and vison assistant for biomedicine (LLaVa-Med). Other visual-text generative models can be employed.

In an embodiment, predicted healthcare answers 532 and unanswerable questions 533 can be prompted to a decision-making entity 628 to obtain confirmed answers 537 by employing a prompter 535. Accuracy of the predicted healthcare answers 532 can be ensured by confirming with the predicted healthcare answers with the decision-making entity 628. In an embodiment, the prompter 535 can show a list of predicted healthcare answers 532 and unanswerable questions 533 which can be confirmed and answered by the decision-making entity 628.

In an embodiment, the prompter 535 can show the list of predicted healthcare answers 532 and unanswered questions 533 to a display. In another embodiment, prompter 535 can show the list of predicted healthcare answers 532 and unanswered questions 533 through a network to show the list to a connected device. In another embodiment, the prompter 535 can show a list of predicted summary sentences 534 to a display to be confirmed by a decision-making entity 628.

In block 130, healthcare summary sentences 534 can be obtained by synthesizing, with AI, a sequence of complete sentences from the predicted healthcare answers 532 and the relevant healthcare questions 513.

In an embodiment, healthcare summary sentences 534 can be obtained by synthesizing, with AI, the predicted healthcare answers 532 and the relevant healthcare questions 513 to a sequence of complete sentences by prompting the extractive question answering model 529. The healthcare summary sentences 534 can be saved in computer memory.

In another embodiment, the extractive question answering model 529 can be configured to fill in missing text from the relevant healthcare questions 513 and predicted healthcare answers 532 to complete sentences by using the healthcare data record 501.

In block 140, a healthcare technical report 541 can be generated autonomously from the healthcare summary sentences 534 to assist a decision making of a healthcare professional 601.

In an embodiment, the healthcare technical report 541 can contain a medical summary based on the healthcare data record 501. The healthcare technical report 541 can assist the decision making (e.g., diagnosis, treatment, etc.) of a healthcare professional 601 (e.g., doctor, nurse, etc.) as it can provide accurate healthcare summaries based on the specific healthcare data of a patient. In an embodiment, the technical report 541 can be generated by a report generator 539 (shown in FIG. 3) that compiles the healthcare summary sentences 534 and arranges them according to a specific scheme that can be configured by a decision-making entity 628. In an embodiment, the scheme can mirror the arrangement of the healthcare data record 501. In another embodiment, the scheme can be chronological. In another embodiment, the scheme can be determined based on the chief complaint (e.g., based on symptoms). In another embodiment, the healthcare technical report can be generated autonomously by the report generator 539 with an AI model (e.g., extractive question answering model 529) trained with the healthcare summary sentences 534, preceding context 507, and healthcare data record 501.

In another embodiment, the healthcare technical report 541 can contain the healthcare summary sentences 534 that can be arranged according to the schemes discussed herein.

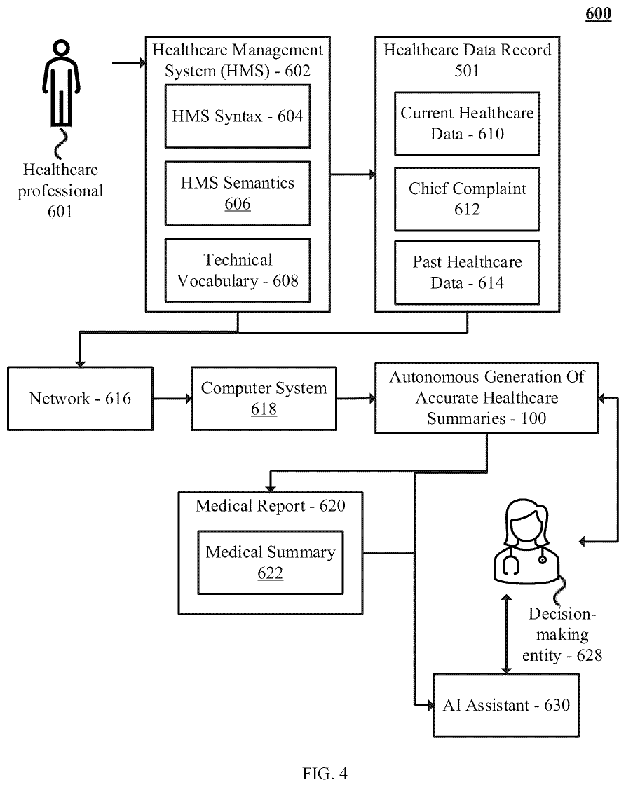

In another embodiment, an AI assistant 630 (shown in FIG. 4) can present the healthcare technical report 541 (e.g., medical report). In another embodiment, the AI assistant 630 can place medical summaries into a workflow (e.g., creating a medical summary document) that the healthcare professional 601 is currently working on.

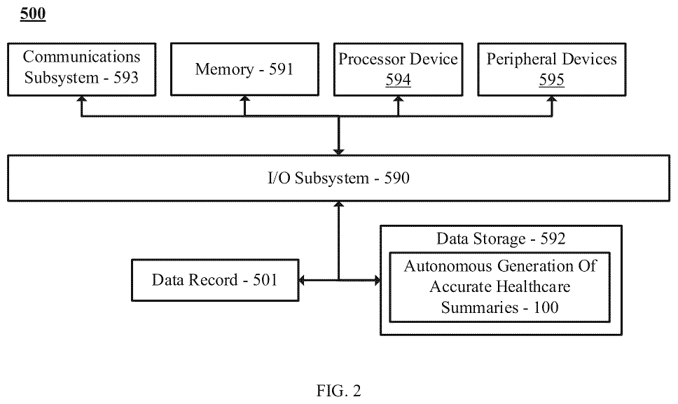

Referring now to FIG. 2, a block diagram for an overview of a computing device that may be configured to autonomously generate accurate healthcare summaries 500, is illustratively depicted in accordance with an embodiment of the present invention.

The computing device 500 illustratively includes the processor device 594, an input/output (I/O) subsystem 590, a memory 591, a data storage device 592, and a communication subsystem 593, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 591, or portions thereof, may be incorporated in the processor device 594 in some embodiments.

The processor device 594 may be embodied as any type of processor capable of performing the functions described herein. The processor device 594 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 591 may be embodied as any type of volatile or non-volatile memory or data storage capable of performing the functions described herein. In operation, the memory 591 may store various data and software employed during operation of the computing device 500, such as operating systems, applications, programs, libraries, and drivers. The memory 591 is communicatively coupled to the processor device 594 via the I/O subsystem 590, which may be embodied as circuitry and/or components to facilitate input/output operations with the processor device 594, the memory 591, and other components of the computing device 500. For example, the I/O subsystem 590 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 590 may form a portion of a system-on-a-chip (SOC) and be incorporated, along with the processor device 594, the memory 591, and other components of the computing device 500, on a single integrated circuit chip.

The data storage device 592 may be embodied as any type of device or devices configured for short-term or long-term storage of data such as, for example, memory devices and circuits, memory cards, hard disk drives, solid state drives, or other data storage devices. The data storage device 592 can store program code for autonomous generation of accurate healthcare summaries 100. Any or all of these program code blocks may be included in a given computing system.

The communication subsystem 593 of the computing device 500 may be embodied as any network interface controller or other communication circuit, device, or collection thereof, capable of enabling communications between the computing device 500 and other remote devices over a network. The communication subsystem 593 may be configured to employ any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 592. The peripheral devices 592 may include any number of additional input/output devices, interface devices, and/or other peripheral devices. For example, in some embodiments, the peripheral devices 592 may include a display, touch screen, graphics circuitry, keyboard, mouse, speaker system, microphone, network interface, and/or other input/output devices, interface devices, GPS, camera, and/or other peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be employed. Moreover, additional processors, controllers, memories, and so forth, in various configurations can also be utilized. These and other variations of the processing system 500 are readily contemplated by one of ordinary skill in the art given the teachings of the present invention provided herein.

Referring now to FIG. 3, illustrating a software program for autonomous generation of accurate healthcare summaries 300, in accordance with an embodiment of the present invention.

In an embodiment, during a training phase, preceding context 507 can be generated from ground truth summaries 508 obtained from a healthcare data record 501 by employing a question generative model 505 and question templates 506. A transformer model 511 can be fine-tuned with the extracted healthcare data 503, the preceding context 507, subsequent questions 509 to obtain a fine-tuned transformer model 512. During an inference phase, relevant healthcare questions 513 can be predicted based on the extracted healthcare data 503 and the preceding context 507 by employing the fine-tuned transformer model 512. Predicted healthcare answers 532 can be predicted from the relevant healthcare questions 513 by employing an extractive question answering model 529. Predicted healthcare answers 532 and unanswerable questions 533 can be prompted through a prompter 535 to a decision-making entity 628 to obtain confirmed answers 537. Healthcare summary sentences 534 can be obtained by synthesizing, with AI, the predicted healthcare answers 532 and the relevant healthcare questions 513 to a sequence of complete sentences. A healthcare technical report 541 can be generated through a report generator 539 by utilizing the healthcare summary sentences 534.

In another embodiment, during the inference phase, summary sentences 534 can be generated by the extractive question answering model 529 based on the extracted healthcare data 503, the preceding context 507, and past summary sentences 536. The summary sentences 534 can be prompted to a decision-making entity 628 to obtain confirmed answers 537. The healthcare technical report 541 can be generated through a report generator 539 based on the confirmed answers 537.

Referring now to FIG. 4, illustrating a practical application of the computer-implemented method of autonomous generation of accurate healthcare summaries 600, in accordance with an embodiment of the present invention.

In an embodiment, a healthcare professional 601 (e.g., doctor, nurse, etc.) can enter a healthcare data record 501 into a healthcare management system (HMS) 602. The healthcare data record 501 can include current healthcare data 610, chief complaint 612 and past healthcare data 614. The HMS 602 can implement their own technical syntax 604, technical semantics 606, and technical vocabulary 608. The healthcare data record 501 can be sent to a computer system 618 that can implement autonomous generation of accurate healthcare summaries 100. through a network 616. The computer system 618 can generate a medical report 620 that can contain medical summary 622 to a decision-making entity 628 (e.g., a healthcare professional).

In another embodiment, the computer system 618 can employ an AI assistant 630 to provide a medical report 620 to the decision-making entity 628 through the workflows (e.g., creating a document of the medical report, etc.) of the decision-making entity 628.

In another embodiment, the HMS 602 can be a data management system for different fields such as educational systems (e.g., student health monitoring, etc.), public sector systems (e.g., community health monitoring, etc.), etc. The technical report 541 can be relevant to such fields. Other practical applications are contemplated.

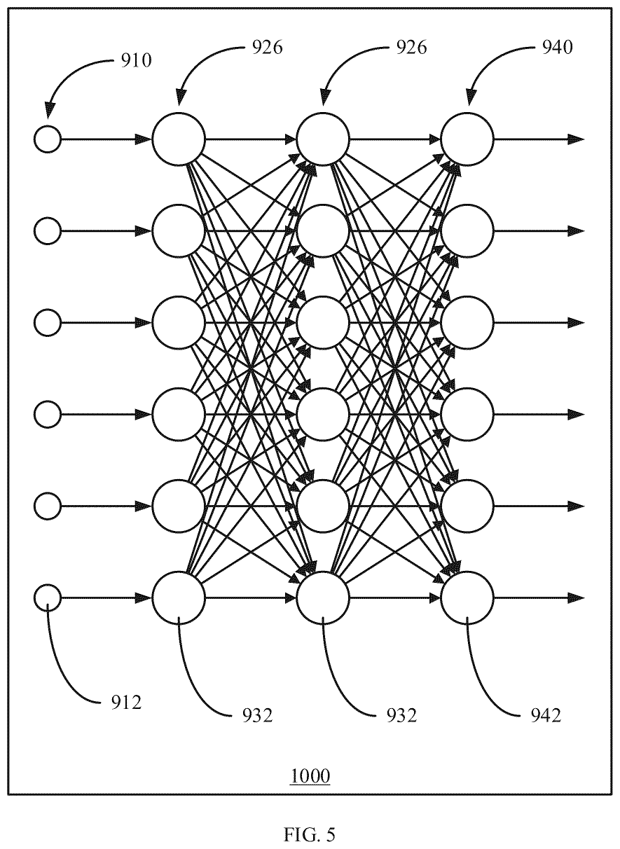

Referring now to FIG. 5, a block diagram showing an overview of deep learning neural networks, in accordance with an embodiment of the present invention.

A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the inputted data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

The deep neural network 1000, such as a multilayer perceptron, can have an input layer 911 of source nodes 912, one or more computation layer(s) 926 having one or more computation nodes 932, and an output layer 940, where there is a single output node 942 for each possible category into which the input example could be classified. An input layer 911 can have a number of source nodes 912 equal to the number of data values 912 in the input data 911. The computation nodes 932 in the computation layer(s) 926 can also be referred to as hidden layers, because they are between the source nodes 912 and output node(s) 942 and are not directly observed. Each node 932, 942 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the inputted data. A deep neural network can be fully connected, where each node in a computational layer is connected to all other nodes in the previous layer, or may have other configurations of connections between layers. If links between nodes are missing, the network is referred to as partially connected.

In an embodiment, the computation layers 926 of the question generative model 505 can generate series of sequences of text based on ground truth summaries 508. The output layer 940 of the question generative model 505 can then provide the overall response of the network to the ground truth summaries 508 and the preceding context 507. In another embodiment, the computation layers 926 of the fine-tuned transformer model 512 can generate series of sequences of text based on the preceding context 507. The output layer 940 of the fine-tuned transformer model 512 can then provide the overall response of the network to the preceding context 507 as relevant healthcare questions 513. In another embodiment, the computation layers 926 of the extractive question answering model 529 can generate series of sequences of text based on an extracted healthcare data 503, and answerable questions 531. The output layer 940 of the extractive question answering model 529 can then provide the overall response of the network to the extracted healthcare data 503, and answerable questions 531 as predicted healthcare answers 532. In another embodiment, the output layer 940 of the extractive question answering model 529 can then provide the overall response of the network to the extracted healthcare data 503, and answerable questions 531 as summary sentences 534.

Training a deep neural network can involve two phases, a forward phase where the weights of each node are fixed and the input propagates through the network, and a backwards phase where an error value is propagated backwards through the network and weight values are updated. The computation nodes 932 in the one or more computation (hidden) layer(s) 926 perform a nonlinear transformation on the input data 912 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs). These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for autonomous generation of accurate healthcare summaries, comprising:
predicting relevant healthcare questions based on a preceding context by employing a fine-tuned transformer model;
predicting answers to the relevant healthcare questions by employing an extractive question answering model that utilizes extracted healthcare data from a healthcare data record to obtain predicted healthcare answers;
synthesizing, with artificial intelligence (AI), complete sentences from the predicted healthcare answers and the relevant healthcare questions to obtain healthcare summary sentences; and
generating a healthcare technical report autonomously from the healthcare summary sentences to assist with a decision making of a healthcare professional.

2. The computer-implemented method of claim 1, further comprising employing an AI assistant trained with extracted healthcare data and corresponding textual prompts for a patient to assist with the decision making of a healthcare professional in generating a medical summary of the patient based on the healthcare summary sentences.

3. The computer-implemented method of claim 1, further comprising fine-tuning a transformer model with ground truth summaries, the preceding context, and subsequent questions to obtain a fine-tuned transformer model.

4. The computer-implemented method of claim 3, wherein fine-tuning the transformer model further comprises converting the ground truth summaries into the subsequent questions by employing a question generative model.

5. The computer-implemented method of claim 4, wherein converting the ground truth summaries into the subsequent questions further comprises utilizing question templates to construct the subsequent questions from extracted entities.

6. The computer-implemented method of claim 1, further comprising prompting the predicted healthcare answers and unanswerable questions to a decision-making entity to obtain confirmed answers.

7. The computer-implemented method of claim 1, further comprising training the extractive answer model to pair answer contexts that involve abbreviations with questions that use spelled out words.

8. A system to autonomously generate accurate healthcare summaries comprising:
a memory; and
one or more processor devices in communication with the memory to:
predict relevant healthcare questions based on a preceding context by employing a fine-tuned transformer model;
predict answers to the relevant healthcare questions by employing an extractive question answering model that utilizes extracted healthcare data from a healthcare data record to obtain predicted healthcare answers;
synthesize, with artificial intelligence (AI), complete sentences from the predicted healthcare answers and the relevant healthcare questions to obtain healthcare summary sentences; and
generate a healthcare technical report autonomously from the healthcare summary sentences to assist with a decision making of a healthcare professional.

9. The system of claim 8, further comprising one or more processor devices in communication with the memory to employ an AI assistant trained with the extracted healthcare data and corresponding textual prompts for a patient to assist a doctor in generating a medical summary of the patient based on the healthcare summary sentences.

10. The system of claim 8, further comprising one or more processor devices in communication with the memory to fine-tune a transformer model with ground truth summaries, the preceding context, and subsequent questions to obtain a fine-tuned transformer model.

11. The system of claim 10, wherein one or more processor devices in communication with the memory to fine-tune the transformer model further comprises one or more processor devices in communication with the memory to convert ground truth summaries into the subsequent questions by employing a question generative model.

12. The system of claim 11, wherein one or more processor devices in communication with the memory to convert the ground truth summaries into the subsequent questions further comprises one or more processor devices in communication with the memory to utilize question templates to construct the subsequent questions from extracted entities.

13. The system of claim 8, further comprising one or more processor devices in communication with the memory to prompt the predicted healthcare answers and unanswerable questions to a decision-making entity to obtain confirmed answers.

14. The system of claim 8, further comprising one or more processor devices in communication with the memory to train the extractive answer model to pair answer contexts that involve abbreviations with questions that use spelled out words.

15. A non-transitory computer program product comprising a computer-readable storage medium including program code to autonomously generate accurate healthcare summaries, wherein the program code when executed on a computer causes the computer to perform:
predicting relevant healthcare questions based on a preceding context by employing a fine-tuned transformer model;
predicting answers to the relevant healthcare questions by employing an extractive question answering model that utilizes extracted healthcare data from a healthcare data record to obtain predicted healthcare answers;
synthesizing, with artificial intelligence (AI), complete sentences from the predicted healthcare answers and the relevant healthcare questions to obtain healthcare summary sentences; and
generating a healthcare technical report autonomously from the healthcare summary sentences to assist with a decision making of a healthcare professional.

16. The non-transitory computer program product of claim 15, further comprising employing an AI assistant trained with the extracted healthcare data and corresponding textual prompts for a patient to assist a doctor in generating a medical summary of the patient based on the healthcare summary sentences.

17. The non-transitory computer program product of claim 15, further comprising fine-tuning a transformer model with ground truth summaries, the preceding context, and subsequent questions to obtain a fine-tuned transformer model.

18. The non-transitory computer program product of claim 17, wherein fine-tuning the transformer model further comprises converting ground truth summaries into the subsequent questions by employing a question generative model.

19. The non-transitory computer program product of claim 18, wherein converting the ground truth summaries into the subsequent questions further comprises utilizing question templates to construct the subsequent questions from extracted entities.

20. The non-transitory computer program product of claim 17, further comprising training a question generative model in reverse with an extractive question answering dataset to predict questions from sentences that answer them.
